(19) United States
(12) Patent Application Publication
(10) Pub. No.: US 2021/0110912 A1
(43) Pub. Date: Apr. 15, 2021

(54) SYSTEMS AND METHODS FOR IMPROVED REPORT COLLABORATION

(71) Applicant: GE Precision Healthcare LLC, Wauwatosa, WI (US)

(72) Inventor: Tiasa Mukherjee, Bangalore (IN)

(21) Appl. No.: 16/599,844

(22) Filed: Oct. 11, 2019

(51) Int. Cl. G16H 30/20; G06F 17/27; G16H 10/60; G16H 80/00

(52) U.S. Cl. CPC G16H 30/20; G16H 80/00; G16H 10/60; G06F 17/274

(57) ABSTRACT

Systems and methods for improved report collaboration are proposed. A reporting system may interact with a reporting assistant. The reporting assistant provides analysis of the report being prepared and provides on-the-fly suggestions for report improvement. The analysis of the report may be improved by using one or more rule engines or learning engines. In the healthcare industry, one type of report may be radiology reports.

### TECHNICAL FIELD

The subject matter disclosed herein relates generally to technology for the creation of digital reports and collaboration between individuals using digital reports, including, but not limited to, radiology reports.

### BACKGROUND

Digital technologies are used often to convey information between individuals, or a collection of individuals. One form of information conveyance is a digital report. One or more people prepare information in a digital report format using reporting technologies, and the report is provided to one or more recipients over the internet or another communication framework.

The most useful reports are those that communicate information in a clear way that the recipient can understand, have all the relevant information for the task or topic at hand, and are provided in a timely fashion. Reports are additionally useful if they can easily leverage the information already available in a related digital information ecosystem, such that there is a higher chance that all useful information related to the report task or topic can be factored in the creation and communication of the report. In healthcare, higher quality reports between healthcare professionals promote higher quality of decision-making and care provided to patients. Technological systems and methods for improved report creation and collaboration are proposed herein.

### BRIEF DESCRIPTION

In accordance with an embodiment, a system is provided that can include an image viewer and reporting system for displaying report information and receiving user input; and a reporting assistant that receives and analyzes said report information and user input in comparison to at least one rule engine to generate a report update action, and providing the report update action to the image viewer and reporting system. Further, one rule engine may be a learning engine. The system may further include a feedback engine; an archive with historical reports; and wherein said learning engine receives historical reports from said archive and uses machine learning to extract features from historical reports to provide update actions related to positive aspects and improvement opportunities of historical reports supplied from the feedback engine. The system may further include a requirements evaluation in comparison to at least one of a legal, financial, healthcare system, industry, care based, or safety requirements.

The report information may include radiology information; and the image viewer and reporting system may display a radiology image. Further, the system may include a recipient preference engine that analyzes the report information in comparison with the preferences of the intended recipient and generates a report update action including a recommendation for altering the report to fit with the preferences of the intended recipient, and wherein the recipient is a patient or a referring physician. Further, the system may include a wording and vocabulary engine that analyzes the radiology report information for at least one of double negatives, defensive posturing, ambiguous vocabulary, hedge vocabulary, modifiers such as quantitative adjectives, and generalizations. The analysis to generate a report update action may include the steps of: analyzing user input and report information to identify report recommendations; performing a requirements evaluation comparing user input and report information with report requirements to identify rule violations; evaluating user input and report information to identify useful output recommendations based on recipient information; and providing an update action to the image viewer and reporting system, said update action including at least one of a report recommendation, rule violation, or useful output recommendation. And the at least one report engine in such an embodiment can comprise at least one rule engine related to receiving input; at least one rule engine related to processing and analyzing input; at least one rule engine related to requirements evaluation; and at least one rule engine related to useful output evaluation.

In accordance with an embodiment, systems, methods, and computer readable mediums are proposed that may include the steps of: receiving user input and report information from an image viewer and reporting system; analyzing user input and report information to identify report recommendations; performing a requirements evaluation comparing user input and report information with report requirements to identify rule violations; evaluating user input and report information to identify useful output recommendations based on recipient information; and providing an update action to the image viewer and reporting system, said update action including at least one of a report recommendation, rule violation, or useful output recommendation. The systems, methods, and computer readable mediums may further include the step of displaying said update action on a user interface of the image viewer and reporting system. The systems, methods, and computer readable mediums may further include the steps of receiving user input response in response to the displaying of the update action on the user interface of the image viewer and reporting section; and modifying the report information based on said user input response. The update action may be a useful output recommendation and said update action is applied automatically to the report without user interaction. Further, each of the receiving, analyzing, performing, and evaluating steps may include at least one rule engine to process the report information and output update actions.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 shows a system for report creation and collaboration, according to an embodiment.

FIG. 2 shows a process and system for medical care, according to an embodiment.

FIG. 3 shows process and system for improving report creation and collaboration, according to an embodiment.

FIG. 4 shows a process and block diagram for an improved healthcare reporting and collaboration technology, according to an embodiment.

FIG. 5 shows a process and block diagram of an example rule engine, according to an embodiment.

FIG. 6 shows a process for a rule engine, according to an embodiment.

FIG. 7 shows a process and block diagram for rules in a rule engine, according to an embodiment.

FIG. 8 shows another process and block diagram for rules in a rule engine, according to an embodiment.

FIG. 9 shows a process for rule authoring, according to an embodiment.

FIG. 10 shows a learning engine, according to an embodiment.

FIG. 11 shows an example report, according to an embodiment.

FIG. 12 shows a user interface for an improved reporting and collaboration technology, according to an embodiment.

FIG. 13 shows another user interface for an improved reporting and collaboration technology, according to an embodiment.

FIG. 14 shows a schematic block diagram of a sample-computing environment, according to an embodiment.

FIG. 15 shows a schematic block diagram of another sample-computing environment, according to an embodiment.

FIG. 16 shows a schematic block diagram of another sample-computing environment, according to an embodiment.

FIG. 17 shows a schematic block diagram of another sample-computing environment, according to an embodiment.

FIG. 18 shows a schematic block diagram of another sample-computing environment, according to an embodiment.

FIG. 19 shows a schematic block diagram illustrating a suitable operating environment, according to an embodiment.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized, and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

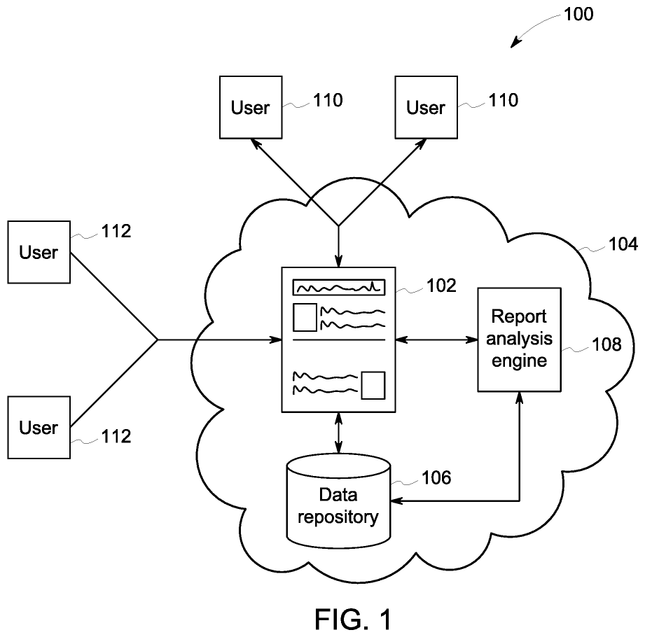

FIG. 1 shows a system for report creation and collaboration, according to an embodiment. FIG. 1 includes collaboration ecosystem 100, digital report 102, communication framework 104, data repository 106, report analysis engine 108, user 110, and user 112.

One or more users 110 collaborate with one or more users 112. One group of users may prepare information for the other party to receive or they may work together on the report content. Report content may include many forms of communication, including text, images, graphics, videos, voice output, and other forms of inter-human digital communication. Data repository 106 stores data, including the reports, system information, and other information. Report analysis engine 108 provides an improved experience for the users of the system by reviewing submitted information, correcting errors, providing suggestions, and formatting the final report in the form most useful to the end recipients of the report, as will be discussed further herein. Communication framework 104 consists of one or more communication protocols to allow various parties to digitally communicate with each other and with report analysis engine and data repository. This may be the internet, a local network, wireless short-range technologies, or other communication frameworks.

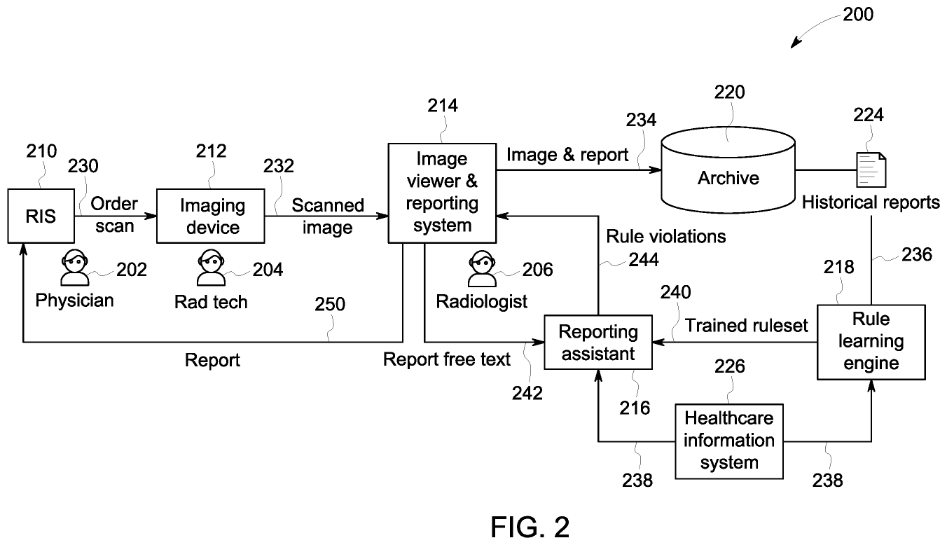

FIG. 2 shows a process and system for medical care, according to an embodiment. FIG. 2 includes medical care ecosystem 200, physician 202, radiology tech 204, radiologist 206, radiology information system (“RIS”) 210, imaging device 212, image viewer and reporting system 214, reporting assistant 216, rule learning engine 218, archive 220, historical reports 224, healthcare information system 226, order scan 230, scanned image 232, image and report 234, historical reports transfer 236, healthcare information transfer 238, trained ruleset 240, report free text 242, rule violations and improvements 244, and report 250.

The process and system of FIG. 2 shows a radiology example of report creation and collaboration. Radiologists specialize in diagnosing and treating injuries and diseases using images acquired via medical imaging systems. Radiologists may be trained to use medical imaging systems to acquire medical images, or may work with imaging technologists who are specially trained to control medical imaging systems to acquire medical images. Radiologist review and interpret medical images to determine a diagnosis.

The reports prepared by radiologists are important in the review and decision making for medical care. The better the reports, the higher quality of care, lower cost of care, and time saved. Radiology reports are an important means of communication between radiologist and physician. Further, radiology reports are influenced by the personal preferences and experiences of the radiologist; consequently, there is a large individual stylistic component. There are variations of preference of reporting style amongst referrers. Close collaboration with primary care physicians will produce reports that meet their needs and thereby help to improve the quality of care.

Radiology reports are medico-legal documents. Poor communication has been found to be a causative factor in the lawsuits involving radiologists. Further, such reports may need to meet institution, insurance, regulatory, and other legal requirements. Preventing mistakes may reduce liability, help process payments for medical procedures, and help governments process medical data.

While the use of templates and trainings are methods to achieve better communication, they are not automated through digital technologies in the way that reports can be customized per physician specific preferences during the report creation (in real time) and further outline and remind the radiologist on what to keep in mind while generating same report for two different referring physicians.

In FIG. 2, Physician 202 may be a referring or primary care physician who interacts with a patient to diagnose a medical issue. Through information system 210 they order an imaging scan of a patient. Radiology tech 204 uses imaging device 212 to image the region of interest of a patient to produce scanned image 232. A radiologist uses image viewer and reporting system to review scanned image 232 and provide a report 250 back to the physician 202. Such reports are also saved to archive 220 along with the related scanned image 232.

In an embodiment, technology is introduced to improve the process of radiologist 206 in creating the report 250, discussed further herein throughout. Reporting assistant 216 works with image viewer and reporting system 214 to review the information put into the report and to provide back rule violations and report improvements. This review of information in the report may leverage trained rulesets 240 from rule learning engine 218 to identify areas of violation and improvement to provide the radiologist. Such rulesets may be programmed in the system (subject to future ruleset updates) or may be dynamically adjusted using artificial intelligence technologies such as machine learning or deep learning. Rule learning engine 218 analyses data from the report free text 242, health information system 226, and historical reports 224. Reporting assistant 216 may also provide additional helpful information for the report, which may be auto-populated or suggested for inclusion into report 250. This information may come from rule learning engine 218 as well as health information system 226. Health information system data may include electronic medical record data, appointment data, regulation data, institution requirements data, physician preference data, and many other types of data related to healthcare and finance, as further discussed hereinbelow.

Free-text reporting is a common practice in radiology. There are no set standards when it comes to reporting style. There are some common guidelines to bring uniformity, however, physicians look for customized reporting styles which may be different from the reporting radiologist's documentation style. For example, some physicians prefer narrative report while others prefer bullet points in the observation and assessment section of the report. Also, some physicians prefer conclusive assessment while others are fine with pending correlation data. Reporting assistant 242 can analyze the prospective recipients of the report and provides suggestions for formatting based on the recipient preferences.

The technology of FIG. 2, and herein throughout, helps to solve issues related to whether the documentation method preferred by the physician 202 is different from that of the reporting radiologist 206, preventing an issue of interpretation challenge, saving time in reading and understanding the same report, prevent lag in the care continuum, and/or prevent incorrect diagnosis (due to biases) or dissatisfaction. The technology of FIG. 2, and herein throughout, bridges the gap between expected and actual style of reporting between the radiologist and the referring physician. Thus, the technology of FIG. 2 helps in collaboration between individuals on reports.

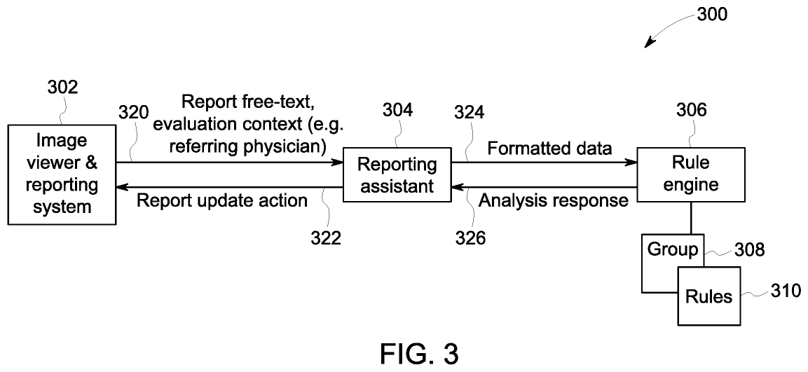

FIG. 3 shows process and system for improving report creation and collaboration, according to an embodiment. FIG. 3 includes report enhancement system 300, image viewer and reporting system 302, reporting assistant 304, rule engine 306, rule groups 308, individual rules 310, radiologist input 320 such as report free-text and evaluation context, report update action 322, formatted data 324, and analysis response 326.

FIG. 3 shows such a system for generating a report, which may include an image viewer and reporting system for displaying report information and receiving user input; a reporting assistant for analyzing said report information and user input, and providing a report update action to the image viewer and reporting system; wherein said analyzing compares the report information with at least one rule engine to provide an analysis response.

Image viewer and reporting system 302 is a software and hardware system that allows the individual generating the report to view one or more related medical images, input their diagnosis and other user input, and also receive report suggestions, update actions, and on-the-fly reminders for report improvement from reporting assistant 304. The report suggestions may be content suggestions for including such as patient history, industry trends, and other relevant information as determined based on rule engine 306 based on healthcare information system information, historical reports, and other data.

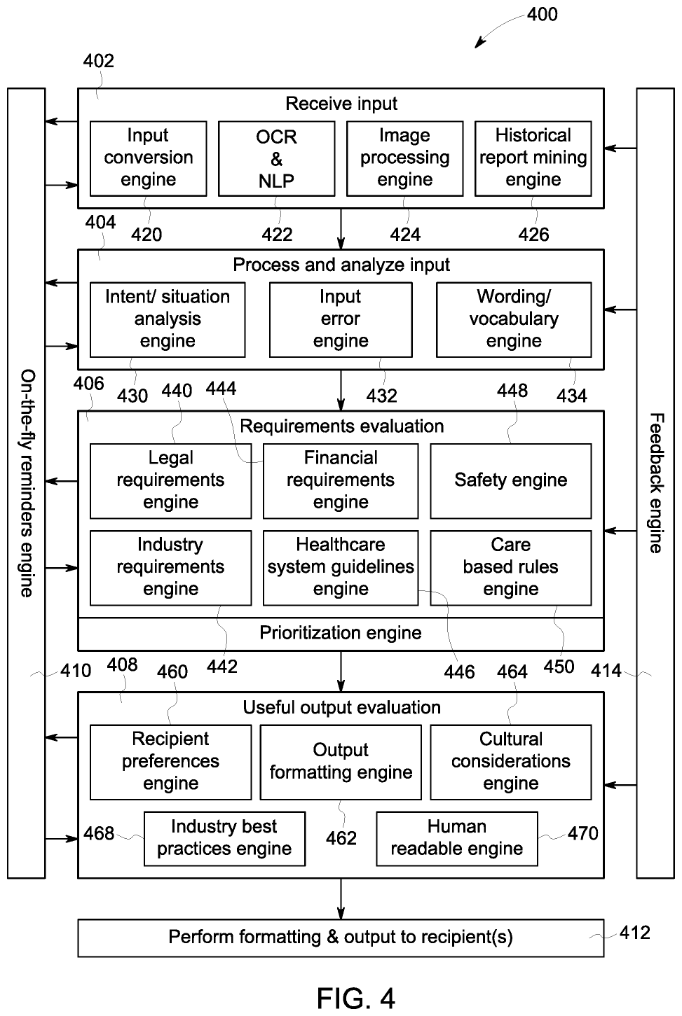

Reporting assistant 304 utilizes a reporting module and rule engine 306. Rule engine 306 processes formatted data 324 through one or more evaluation engines—as discussed further in relation to FIG. 4—to generate analysis responses 326. This may include validating a portion of the free text report against an evaluation engine. Evaluation engines can include rule groups 308 and individual rules 310. Rule engines may consist rules output from a preconfigured listing, pre-programmed analysis software, or decision trees. Rule engines may also be dynamically computer generated from a learning engine, which may be powered by machine learning, deep learning, or other artificial intelligence technology. Rule engines may also be a hybrid of a pre-programmed analysis software with parts that are dynamically computer generated from a learning engine. Rule engine 306 determines one or more analysis responses 326 and sends the analysis responses 326 to reporting assistant 304. Reporting assistant 304 evaluates the one or more analysis responses 304 to provide a report update action 322, which may include an on-the-fly reminder, content to suggest including in the report, rule violations, output formatting suggestions, and further ways to edit the report as discussed herein throughout. Image viewer and reporting system then applies the report update action to the report and a visualization in the report viewer user interface. The reporting system user interface can display the report update action or on-the-fly reminders in the form of icons, prompts, and violations around the area of interest in the report. This allows the user to receive report improvement suggestions and incorporate the changes during the report authoring and generation.

FIG. 4 shows a process and block diagram for an improved healthcare reporting and collaboration technology, according to an embodiment. FIG. 4 includes report analyzer technology 400, receive input step 402, process and analyze input step 404, requirements evaluation step 406, useful output evaluation step 408, on the fly reminders engine 410, perform formatting and output to recipient(s) step 412, feedback engine 412, input conversion engine 420, optical character recognition (“OCR”) and natural language processing (“NLP”) engine 422, image processing engine 424, historical report mining engine 426, intent and situation analysis engine 430, input error engine 432, wording and vocabulary engine 434, legal requirements engine 440, industry requirements engine 442, financial requirements engine 444, healthcare system guidelines engine 446, safety engine 448, care based rules engine 450, a prioritization engine, recipient preferences engine 460, output formatting engine 462, cultural considerations engine 464, industry best practices engine 468, human readable engine 470. Not all of the steps and engines shown in FIG. 4 are required to be present in the technology system and can be intermingled in various embodiments.

FIG. 4 shows a process improving reports, which may include receiving user input and report information from an image viewer and reporting system; analyzing user input and report information to identify report recommendations; performing a requirements evaluation comparing user input and report information with report requirements to identify rule violations; evaluating user input and report information to identify useful output recommendations based on recipient information; and providing an update action to the image viewer and reporting system, said update action including at least one of a report recommendation, rule violation, or useful output recommendation.

In step 402, the system and methods herein receive the inputs from various sources and prepares it for analysis, which may leverage various engines. The engines shown within steps 402, 404, 406, and 408 are for example in one embodiment, but the engines throughout FIG. 4 may be configured in different ways or not included per various embodiments. Input is received from both human interaction and technological interaction.

Input conversion engine 420 converts human input into a format for the system to best process. Input conversion engine may work together with OCR & NLP engine 422 as needed to understand the input received. Input may be from keyboard, mouse, touch screen, voice, gesture, eye tracking, or other input technologies. Input conversion engine 420 may directly prompt the user for additional information through on-the-fly reminders engine 410 if the input received is not being processed correctly or if the system is undetermined as to a specific input. For example, in the medical sphere, many words are uncommon to standard vernacular and may be pronounced differently by different people. Input conversion engine 420 may ask the user for final decision if it needs to finalize correct input of some information. Input conversion engine 420 may be a learning engine that continuously learns the input styles and linguistics of the users it frequently interacts with. Learning engine 218 receives information from healthcare information system 226, which can include specific users, user preferences, and other information about them. A usage history may be logged per each user helping to enable such continuous learning for input engine 420.

OCR and NLP engine 422 receives input from various sources and converts it to text for inputting into a report. Optical character recognition converts images of typed, handwritten or printed text into machine-encoded text, whether from a scanned document, a photo of a document, a previous report, a scene-photo or from subtitle text superimposed on an image. Natural language processing receives natural human language and converts into machine-encoded text for use in reports.

Image processing engine 424 receives scanned images 232 and processes the image to gain information that may help the report. For example, suites of post-processing software may be leveraged, some with artificial intelligence engines. An initial software diagnosis may be performed to provide the radiologist or other reviewer with some initial suggestions as to regions of interest or suspected medical issues within the image. Such software may identify body structures such as bronchi and blood vessels, give a percentage likelihood that a condition such as cancer may be present, and/or provide overlays of information on the screen to provide additional information to re reviewer. These suggestions may be output to the user to the image viewer and reporting system 214 through the on-the-fly reminders engine 410.

Historical report mining engine 426 reviews the historical reports in archive 220 as well as throughout healthcare information system 238. These reports may be specific to the patient involved in the exam to help determine patient history and past analysis. In addition, the reports reviewed may be related to the referring physician to determine their preferences or the radiologist generating the report to determine past issues or positive feedback. Further, the reports reviewed may be others in the system that were working to review the same type of situation (such as bone cancer or pneumonia or other medical issue). Thus, the historical report mining engine 426 leverages artificial intelligence technologies to continuously learn about the best reports based on the reports reviewed. Feedback engine 414 can be very useful in such learning. If a certain radiologist had positive feedback for certain of their reports, historical report mining engine 426 can identify and share such information. And in some embodiments, historical report mining engine 426 can pre-populate and format the report based on the positive aspects of the reports reviewed, based on the feedback from feedback engine 414. Further, by reviewing previous reports, historical report mining engine 426 can identify certain rules and standards regarding formatting and layout. These rules and standards, in some embodiments, can be run through a validation workflow for review and approval of the user before they are applied to the system automatically moving forward. As an example, historical report mining engine can notice errors from previous reports for a user that can be avoided in the current report. Through pre-formatting and/or on-the-fly reminders, these errors can be avoided thus saving time for the users by preventing rework and/or miscommunications.

In step 404, the systems and methods herein process and analyze the input. Input is received from step 404, and an initial report template is output to the user for interaction. This initial report template may be pre-populated, filled out in certain places, and have other suggestions for the user. This initial report template is sent from reporting assistant 216 to image viewer and reporting system 214 for the user to interact with the report template to start working on the report. The user's analysis and input is further received throughout the report generation process.

Intent and situation awareness engine 430 identifies situational factors that may affect how the report is generated. Intent and situation awareness engine 430 analyzes the appointment record, the input request from the referring physician (or the report requester if not a medical report), related EMR (electronic medical record), and/or other information in the healthcare information system 238 to identify the purpose (intent) of the report request. The initial request may include a specific field or reason for the request, but the supplemental records such as the appointment record and EMR may add additional details that will help a radiologist (or the report preparer if not a medical report) provide the best report, which may include clearly stating the specific detail that allows the referring physician to make a choice between two possible treatments for a clinical condition. These additional details are part of the understanding not only of the intent of the request, but the full situation. Further, if the final report does not include information specifically targeting the intent of the report request, then the reporting assistant 216 can provide an on-the-fly reminder for the report to include such a response related to the intent.

Input error engine 432 detects errors related to the input from step 402. Input errors could be the wrong date, name, body part, or other errors by the user working on the report. Additional errors may come from the automatic report information, potentially that pulls in information from the wrong historical report or the wrong patient. If a potential error is detected, the system can notify the report preparer for review and correction, if needed.

Wording and vocabulary engine 434 analyzes the wording and vocabulary input to the report. Correct spelling and grammar are included in the analysis. Medical dictionaries and word checking compared to the intent of the report are analyzed. For example, if the scanned image 232 and report request are related to the head of a patient, and a word in the report is related to the foot, the wording and vocabulary engine 434 may send an on-the-fly reminder to the report preparer for clarification. Certain terms are interpreted differently by clinicians than by radiologists. For example, clinicians often understand “collection” to mean “abscess.” Unless this is the intended impression, terms such as “fluid accumulation” or “fluid filled structure” may be preferable.

Wording and vocabulary engine 434 also analyzes for issues specific to radiology reports such as: double negatives, defensive posturing, ambiguous vocabulary, hedge vocabulary, modifiers such as quantitative adjectives, and generalizations. Ambiguous and hedge vocabulary are words and phrases are either superfluous to the overall message in the report or open to interpretation by the reader. Modifiers are adjectives without predefinition, and the wording and vocabulary engine 434 can output an on-the-fly reminder that words and phrases should be used only if there are clear definitions in the literature regarding their meaning. Defensive posturing may be indicated by overused of terms like “clinical correlation needed” and “if clinically indicated”. Terms like “clinical correlation needed” and “if clinically indicated” are used to indicate that inadequate clinical information was available to the radiologist during the report creation to make a definitive assessment of the case and additional clinical information (such as lab test results) is required to infer the diagnosis. However, some radiologists overuse these terms, which wording and vocabulary engine 434 can detect and provide an on-the-fly recommendation to the user to evaluate their choice of terminology.

In step 406, the systems and methods herein perform a requirements evaluation. Requirements may include laws, regulations, guidelines, recommendations, and other forms of standardized protocols of communicating information in the related report types. Requirements evaluation step 406 also has a prioritization engine that will decide which requirements take precedence when multiple requirements may exist for a similar field or section of a report. Through receiving on-the-fly reminders and interacting with requirements evaluation engines, as discussed in detail below, a report preparer is trained and improved in their preparation of reports, which can save, time, money, and improve patient care.

Legal requirements engine 440 analyzes the input data and report in comparison to the related country, state, and local laws and regulations. For example, in the healthcare space, understanding the legal implications of radiology reports will enable radiologists to develop strategies for avoiding malpractice suits and stay within the bounds of the law. Legal requirements engine 440 generally outputs requirements for the report that are of higher priority than other requirements for the prioritization engine. For example, a referring physician may like to see a report in a certain style as output from the recipient preferences engine 460, but if that conflicts with a legal requirement the prioritization engine will recommend to the user the legal requirement take priority in an on-the-fly reminder. And in some institutions, they may not allow an option to the report preparer in that circumstance. Additional legal requirements may be how certain reports are coded and submitted for government programs such as Medicare in the United States.

Industry requirements engine 442 analyzes the input and report in comparison to standards or requirements of the related industry, which in some embodiments is healthcare. These requirements may state how radiology reports, or other reports, should be organized and structured across the industry. The industry requirements engine 442 thus not only reviews the content of the report being prepared, but also the structure, formatting, and section layouts. For example, “Observations” section lists the radiologist's observations and findings regarding each area of the body examined in the imaging study, while in the “Impression” section, the radiologist provides a diagnosis. In a free-text report, the sections can be in any order. Industry standards may outline choice for customization such as the order of findings section first and then the impressions section, since the impression combines the findings (and other clinical details like, patient history) to provide the diagnosis. Following an industry standard for reporting may more efficiently convey the most sought for information from the report. Industry requirements engine 442 also may output to the user a suggestion that includes an industry recommendations, not required standards, such as a generally accepted solution is to list the top differential diagnoses and favor the one that is most likely. Another example of an industry recommendation is that a radiology report should include demographics, relevant clinical information, clinical issues, comparison studies and reports, procedures and materials, potential limitations, findings, and overall impression, and thus industry requirements engine 442 may check a report for all of these sections in its analysis.

Financial requirements engine 444 analyzes the input and report in comparison with financial requirements, such as from the insurance industry or government payors. Payors may have required sections to be filled in for reports. In one example, reports should be of sufficient clarity to avoid rescans. Rescanning an image can have cost and insurance implications. And if the recommendation in a report is to order a rescan the reasons should be very clear such that the single rescan solves the issue. For example, a recommendation for a “repeat study (with improved bolus timing and possible sedation to avoid motion artifact) within a definite time frame” can be sent from financial requirements engine 444 which forces clarity of timing and structure to the rescan. Financial requirements engine 444 can define conditional rules that, depending on the mentioned limitation of the current scan, determine the sufficiency of the reason for repeat scan that is documented in the report and alerts the radiologist for need of additional information, in one or more embodiments. In the example above, a condition is defined to check if the reason for rescan in the report has qualifiers similar to “obscured by artifact” or “does not involve the region of interest”, in which case, if the condition check succeeds, no further rules are applied to alert the radiologist. Another rule defined could check qualifiers similar to “poor contrast opacification” and if that condition check succeeds, financial requirements engine 444 would evaluate another condition that checks if the recommendation for the repeat study has the phrases that suggest improved image parameters pre-defined for such limitations.

Healthcare system guidelines engine 446 analyzes the input and report in comparison to guidelines or requirements set up by the healthcare institution or system that the medical care is being provided under. Certain institutions that want to apply standard practices of general good documentation of reports across the institution, can enforce that with this engine by defining a set of common rules. These rules can originate from process improvement discussions on improved collaboration between radiologist and physician, or training needs for new radiologists. If there are common physician preferred rules across all physicians, those can also be advanced to the common “institution requirement” pool. While the use of templates and trainings are some of the methods to achieve better reports and communication with the referring physician, these are not automated, whereas the healthcare system guidelines engine 446 can remind a new radiologist on what to keep in mind while generating the report in real time. And the institution may also have guidelines about the report formatting or quality standards. The institution may enforce uniformity in documentation practices, like mandate sections to be present in a report, irrespective of the physician preference.

Safety engine 448 analyzes the input and report in comparison with potential safety violations. Poorly worded or ambiguous radiology reports may result in negligence on the part of the interpreting radiologist or may lead to errors by referring physicians that result in potential harm to the patient. Safety engine 448 also analyzes the input and report in comparison with safety issues that have occurred in the institution that can be classified as “rule-based” errors, such as a finding that was not reported because a radiologist forgot to open a series of images versus a finding that was not reported because the radiologist saw the finding but underestimated its clinical implications. The data from the health information system 238 is can be important to the continuous learning aspect of the safety engine 448 as other errors throughout the system and elsewhere in the healthcare industry can be learned from to help safety engine 448 best detect potential safety issues.

Care based rules engine 450 analyzes the input and report in comparison with rules groups specific to the type of care detected by intent and situation analysis engine 430. In an example, a “care based” rule group for suspected stroke could consist of a rule that looks for the type of stroke (examples hemorrhagic stroke or thrombotic stroke) in the “inference” or “assessment” section of the report. The absence of such inference would trigger the rule engine to alert the radiologist for missing important details. For certain types of care and medical conditions, care based rules engine 450 stores and evaluates the types of information that the report is likely to contain.

Prioritization engine is shown within step 406 as an example in an embodiment. More generally it is a separate engine that arbitrates which requirements or suggestions output by other engines take priority, or override, for the user to see and interact with when the requirements or suggestions are related to the same field or section of the report. Prioritization engine initially is programmed with rule sets, and then receives feedback from feedback engine 414 as to the selections made by the users of the system and performs learning to alter priority accordingly moving forward. Initial rules are grouped into rule groups and the groups have an override order. For example, physician-style based rule-set can override the organization wide care-based ruleset, based on the override order defined for the rule-group.

In step 408, the systems and methods herein perform a useful output evaluation. The output of a report may be altered based on recipient preferences, the physical device the report is output to, and based on other aspects of the situation such as culture, industry, and human readability.

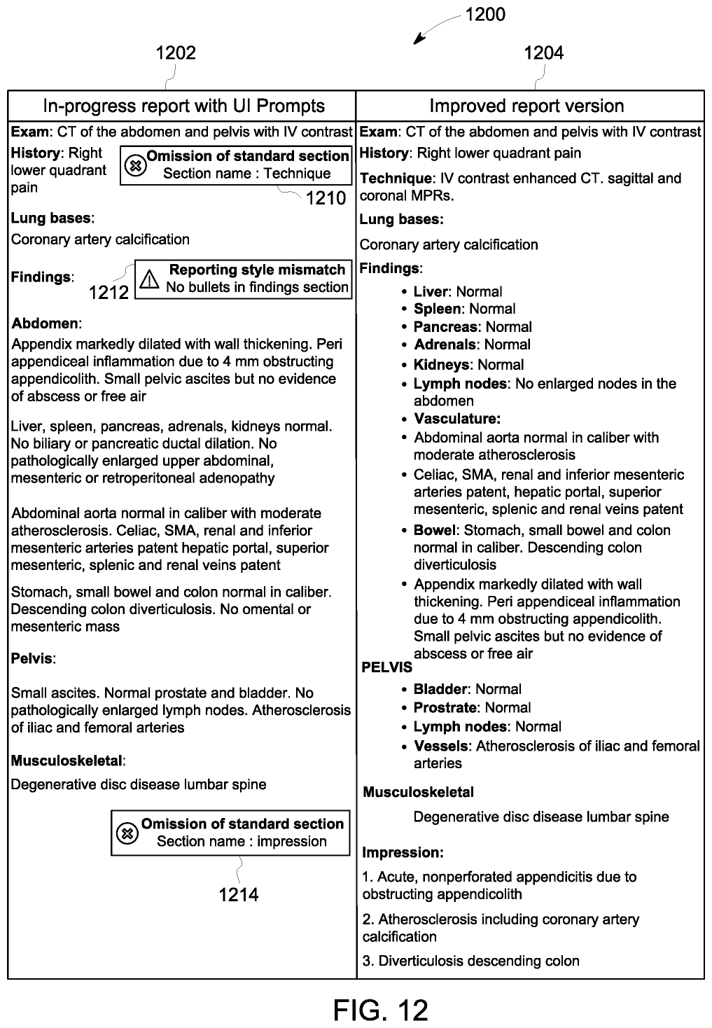

Recipient preferences engine 460 analyzes the input and report and provides suggested updates and improvements to the report based on the specific recipients of the report. In a radiology context, the main recipient is the referring physician 202. Making the report clear and useful to referring physician 202 is of utmost importance for patient care. The referring physician's preferences related to reports may be from a survey, from artificial intelligence learning of their feedback from previous reports and interactions with image viewer and reporting system 214, or directly putting their preferences into the system. Such surveys of the referring physician can be paper forms, computer forms, voice-controlled interactions, human surveys, or other ways to ascertain their preferences. Each report preparer, or radiologist in some examples, may have their own reporting style. Recipient preferences engine 460 helps to standardize the report such that the individual recipient sees similar report styles even from different radiologists. This automation may also save the radiologist time as they do not have to worry as much about the referring physician preferences as the system can either provide on-the-fly reminders on how to adjust the report or can automatically adjust the report in the final output and sending of the report to the recipient. One example on-the-fly reminder is shown in FIG. 12 denoting the reporting style mismatch 1212. For example, recipient preferences engine 460 may have rules that check for presence or absence of bullets based on the physician who ordered the scan. Additional rules can check the length of the report or the presence of conclusion in the report and alert the radiologist depending on the preference configured for the respective referring physician.

Additional recipients may have preferences as well. Patients may prefer a radiology report to be simplified down to a summary with the details moved to a lower portion. Hospital administrators may focus on the key sections that cause the most error or success and these could be highlighted or otherwise marked by recipient preferences engine 460.

Special rules can be configured for reports that are being authored for multidisciplinary meetings (teleradiology) such as tumor boards that are opposite to the concept of “physician customized” reporting rules, which is customized for a specific physician; whereas in this case, it is for mixed audience who may have to discuss and come up with a common set of customizations, which may be specific to the clinical condition.

Output formatting engine 462 analyzes the technology to be used for outputting the report. Depending on the output technology the system may provide formatting recommendations as on-the-fly reminders or may automatically reformat the report. For example, if the technology is a handheld computing device such as a smartphone, the radiology report may move some of the data about the record information down to allow the summary and findings be more clearly displayed at the top. In addition, the report may be displayed through the software packages of multiple electronic medical record (“EMR”) vendors for example, and output formatting engine 462 may be required to format the reports to make them best read for those software packages.

Cultural considerations engine 464 analyzes the input and report in comparison with aspects of the particular geography and culture of the report recipient(s). The subject of informing patients of the results of their imaging examinations varies from culture to culture. In countries where patients would like to receive their results from the radiologist directly, the approach for informing the patient can be configured as a rule. For example, based on the condition if the report is normal and the software is running in a country where radiologist is expected to convey the result, a standard action can be prompted to the radiologist that reminds them of this.

Industry best practices engine 468 analyzes the input and report in comparison with industry best practices. These are not formal standards or requirements but best practices based on actual medical practice in the industry. This may be from analyzing radiology reports and feedback engines from other institutions throughout the industry. This allows the systems and methods herein to start implementing improved practices before they are set forth in requirements or recommendations. Further, these improved practices can be presented to actually change the report guidelines of the respective institution.

Human readable engine 470 analyzes the input and report to improve readability and clarity. Human readable engine 470 may analyze the words in the report and suggest alternate words and phrasing to make it easier to read. Human readable engine 470 may also review the white space to text ratio and offer suggestions to balance out the report such that it is easier to read. This may include more paragraphs, more bullets, or less dense text areas.

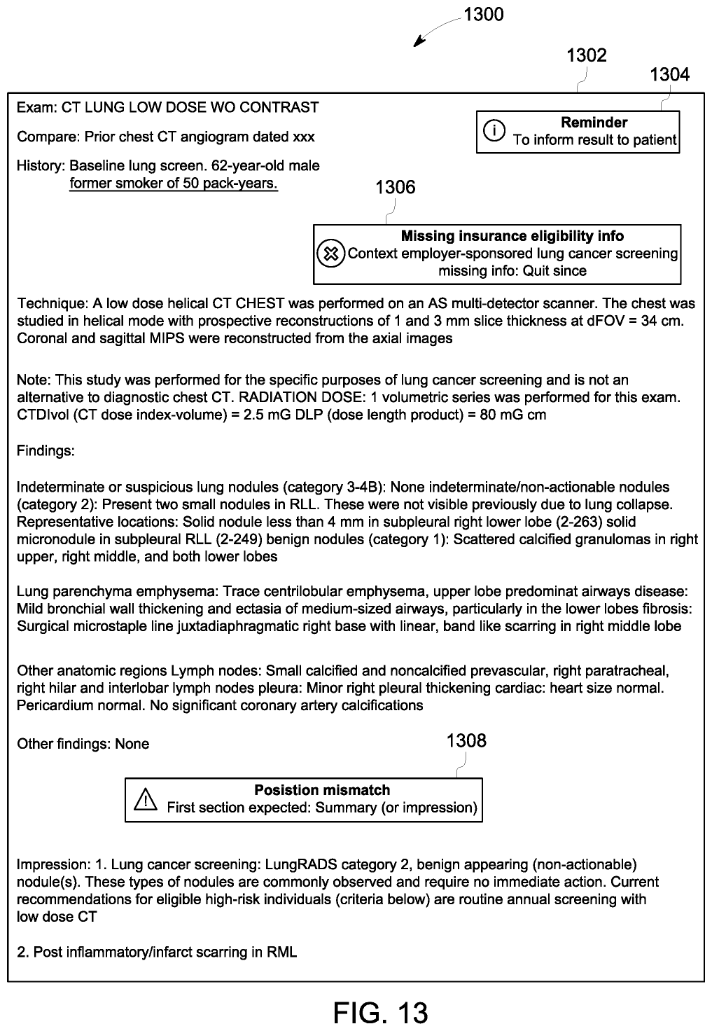

In step 410, an on-the-fly reminders engine receives information from the various steps and engines to provide the user interface user prompts, reminders, and other interactive elements to allow for on-the-fly improvement of the report. Examples of on-the-fly reminders are discussed herein throughout. In addition, on-the-fly reminders may both outline violations during report generation and also offer in-place correction with the “auto correct” option or options provided for selection. FIG. 12 and FIG. 13 include examples of on-the-fly reminders.

In step 412, the systems and methods herein perform formatting and output of the current report to one or more recipients. This may be the person working on editing the report, the referring physician, the patient, hospital management, or other individuals.

In step 414, the systems and methods herein receive information and feedback and provides it to the various engines that utilize feedback for improvement. Feedback may come from user interactions with systems and methods herein including image viewer and reporting system 214, from other inputs into the system, from industry groups, from financial groups, from government groups, and other methods if input of feedback in the system as discussed in relation to the various engines. The users may give positive feedback, suggestions, or other feedback related to certain sections of the report or the overall report itself. For example, a report output may have a prompt asking the user to rate or give a ‘thumbs up’ to various sections, which identifies the positive aspects of the report for feedback engine 414. Feedback engine 414 may record every action and click of the users in the system, in an embodiment, to track the most used features, the most read sections of a report, and the most common actions after reviewing the report, among other things.

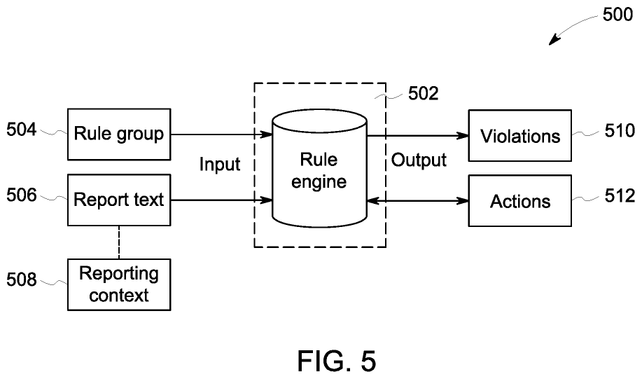

FIG. 5 shows a process and block diagram of an example rule engine, according to an embodiment. FIG. 5 includes rule engine 502, rule group 504, report text 508, report context 508, violations 510, and actions 512. One or more rule groups 504 may exist that rule engine uses in comparison with report text 506 and report context 508 to generate potential violations 510 and suggested or automated actions 512. As an example according to an embodiment, if wording/vocabulary engine 434 is implemented as a hardware or software rule engine, specific rule groups may include groups for double negatives, defensive posturing, ambiguous vocabulary, hedge vocabulary, modifiers such as quantitative adjectives, and generalizations. In this example, if rule engine 502 determines a section of report text 506 uses a double negative, rule engine 502 may output a violation 510 to the reporting assistant or report analysis engine for output to the user for correction. Rule engine 502 may also output an action 512. This action may be a suggested action that the user can choose to take (requiring user to approve) or may be an automated action that changes the report without user action, but a user can undo the action if they choose (requiring user to revert if needed).

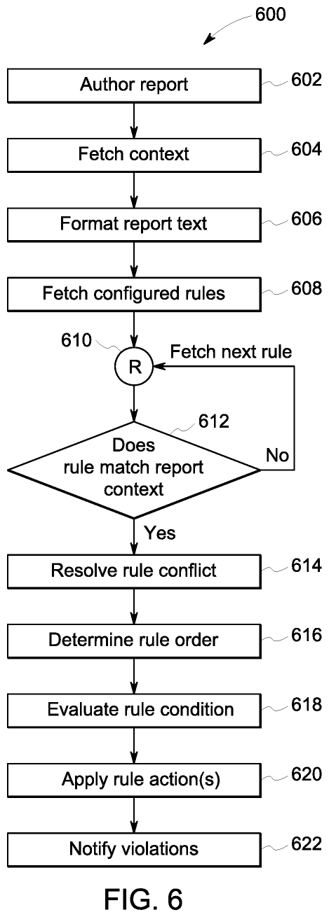

FIG. 6 shows a process for a rule engine, according to an embodiment. FIG. 6 includes rule engine process 600, author report step 602, fetch context step 604, format report step 606, fetch configured rules step 608, rule execution step 610, rule matching decision step 612, resolve rule conflicts step 614, determine rule order step 616, evaluate rule condition step 618, apply rule actions step 620, and notify violations step 622.

In step 602, the user authors the report in conjunction a with image viewer and reporting system. This report-in-progress may be saved by the user periodically or auto-saved by the system. Upon a save or other designated interval, the system can review the newly input material as well as the full scope of material against the rule, learning, and decision engines discussed herein.

In step 604, the systems and methods herein fetch the context related to the report. The context may be related to a certain section of the report, multiple sections, or the full section. For example, if the context of the report is for evaluating for brain abnormalities, this can be pulled from the report quest or from the referring physician's notes to be provided to the radiologist or report reviewer to help them in their evaluation and report preparation. As another example, if the section most recently edited by the user was about the liver of a human patient, the step 604 may review historical reports for that patient to see if the liver was part of any past diagnosis or evaluation which can be provided to the user. Context fetched in step 604 may impact which engines are utilized in analyzing the report.

In step 606, the report text is formatted in such a way to allow for processing by one or more engines. Step 606 may leverage input conversion engine 420, OCR & NLP engine 422, or other formatting conversion requirements that may be specific to the engine that will process the text.

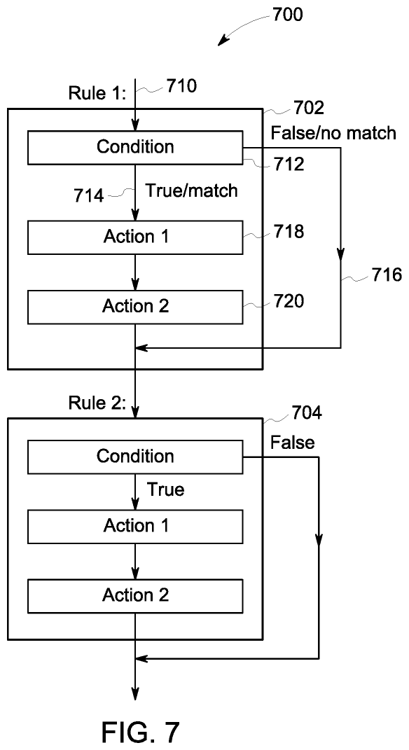

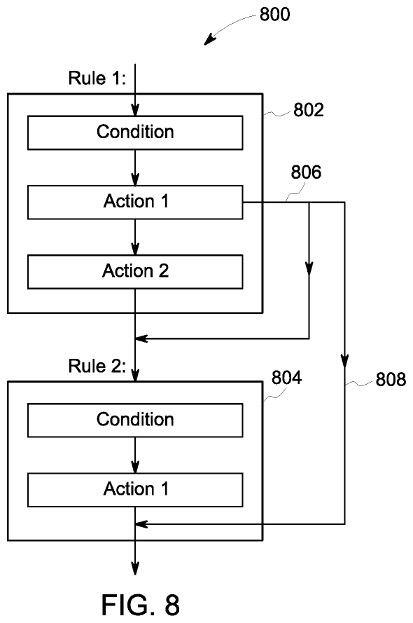

In step 608, the systems and methods herein fetch one or more configured rules. Rule groups may form sets of configured rules, called rulesets. In an embodiment, an individual rule takes in an input and defines what may be allowed or output based on the input. Rules may be based on logic programming. The application of rules to input are additionally discussed with reference to FIG. 7 and FIG. 8.

In step 610, the systems and methods herein apply the input gathered from steps 602, 604, and 606 in series or parallel (which may differ in certain rule engines) with the configured rules fetched in step 608. For each rule or ruleset, step 612 compares the ruleset with the input report and report context information to determine if the rule may match or apply. Thus, step 612 evaluates if certain criteria are met for the ruleset. If no, the system fetches the next rule or ruleset until completion. If yes, the ruleset resolution criteria are invoked, and the systems and methods move to step 614.

In step 614, the systems and methods herein resolve rule conflict. And In step 616, the systems and methods herein determine the rule order. These steps apply when more than one rule may affect a certain portion of the report. Resolving rule conflict manages prioritization when multiple rules may alter a specific portion of text. One rule application may not be needed if another rules' application makes it unnecessary. Determining rule order manages the application order of applying multiple rules. For example, if a sentence is both ambiguous and has double negatives, the change of one issue by applying a rule may already fix the other, so resolving the rule conflict would negate the application of the second rule. And if they do not solve the other issue, step 616 may need to determine in which order the rules should be applied. The rule application may make the most sense to human readers when applied in a certain way. Or the medical accuracy of a recommendation may only be when rules are applied in a certain way. Step 616 may utilize the prioritization engine shown and described in relation to FIG. 4. Also, a rule conflict may simply be a user overriding the suggestion by the system.

In step 618, the systems and methods herein evaluate the rule condition. A rule may have multiple potential actions that can be executed based on certain conditions of the report, report context, or extra information from healthcare information system 226 or other engines. Thus, step 618 selects the appropriate action based on the evaluation of the rule conditions. In some cases, step 618 may trigger one or more other engines based on the rule condition. For example, if the rule condition is related to the healthcare system guidelines, step 618 may query healthcare system guidelines engine 446 for the appropriate rule action to apply.

In step 620, the systems and methods herein apply the rule actions, as discussed also in relation to actions 512. This may be an automatic action or a user prompt giving the user choices on improving the report based on the related rule(s).

In step 622, the systems and methods herein notify the user of violations, as discussed also in relation to violations 510 and on-the fly reminders 410. This is generally through a user prompt on the screen of the image viewer & reporting system, but may also be a text message, email, phone alert, car alert, voice alert, or other type of output to get the attention and feedback from the user generating the report. If the alert or user prompt is not responded to in a certain time, the system may send such alert and user prompts to others such as other radiologists or hospital administrators, especially when the violation may be related to legal requirements or safety.

FIG. 7 shows a process and block diagram for rules in a rule engine, according to an embodiment. FIG. 7 includes rule engine process 700, input 710, rule one 702, rule two 704, condition step 712, true/match 714, false/no match 716, action one 718, and action two 720. In FIG. 7, rules are processed in series, which may occur in some engines. Other engines may process rules in parallel. Input 710 may be the report, partial report text, report context, and/or other information from hospital information system or the reporting assistant. Condition step 712 compares input 710 with the rule conditions. For example, a rule in intent/situation analysis engine 430 may compare the text in the report context report request submission from the referring physician to see if there is a formal reason given for the imaging scan. If false/no match 716, the rule engine moves to the next rule. If true/match 714, the rule engine moves to evaluate related one or more actions, such as action 718 and action 720 in this embodiment. Continuing on with the example, if the reason for the imaging scan is included in the report context, action one 718 may be to alert the radiologist to this information and action two may be to automatically populate the related body part field into the report and action two 720 to make sure that the radiologist addresses the requested concern of the referring physician.

FIG. 8 shows another process and block diagram for rules in a rule engine, according to an embodiment. FIG. 8 includes rule engine process 800, rule one 802, rule two 804, skip actions step 806, and skip rules step 808. FIG. 8 shows a situation where there is an error or other issue when executing an action based on a rule match. This may arise when the action is attempted at the wrong time, cannot execute based on report status, or other issues based on the specific rule engine. In this instance, the actions for the rule may be skipped in step 806. Or, if the rule engine itself is no longer applicable based on the type of error or issue, the remaining rules of the ruleset or the whole rule engine may be skipped in step 808.

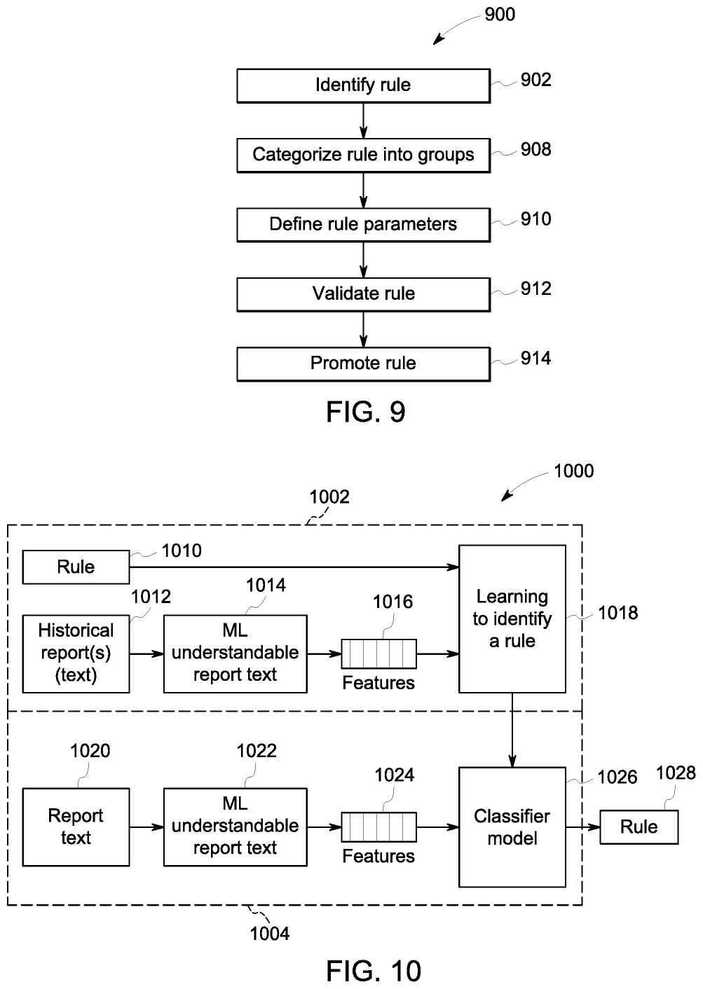

FIG. 9 shows a process for rule authoring, according to an embodiment. FIG. 9 includes rule authorizing process 900, identify rule step 902, categorize rules into groups step 908, define rule parameters step 910, validate rule step 912, and promote rule step 914. Rule authoring may be completed by an authorized person with clinical knowledge to understand rule evaluation outcomes on patient safety. Such an authorized user may create new rules and edit existing rules. In step 902, the systems and methods herein receive the rule from the user and create a new rule record, identifying the rule for the system. In step 908, the systems and methods herein categorize the rule into the proper rule engine (if not pre-selected by the user) and into the associated rule groups.

For example, if a rule is related to the care group oncology, this step associates the rule with care based rules engine 450 and with the ruleset related to oncology. In step 910, the systems and methods herein receive parameters from the user, and adds them to the rule information, such as the triggers and conditions for applying the rule and the actions that the rule takes based on various triggers and conditions. This includes, for example, sequence or order of applying the rule or if the rule is overridable or not. In step 912, the systems and methods herein validate the rule, running a validation process to make sure the rule will execute successfully in the associated rule engine. If not, a prompt may issue to the user for updating the rule accordingly. In step 914, the systems and methods herein promote the rule into the rule engine as a rule to be used in production as part of the associated rule engine. In addition, sometimes rules may be generated in a centralized manner across the industry and promoted to the rule engines all over the industry systems. For example, an employee for the government may generate a new rule related to legal requirements or financial requirements and promote that rule to the associated rule engines of the healthcare systems running those engines.

FIG. 10 shows a learning engine, according to an embodiment. FIG. 10 includes learning engine 1000, training engine 1002, prediction engine 1004, rule 1010, historical report text 1012, machine learning (“ML”) understandable report text 1014, features 1016, learning to identify a rule 1018, report text 1020, ML understandable report text 1022, features 1024, classifier model 1026, and rule 1028. FIG. 10 is specifically adapted as a historical report mining learning engine, in this embodiment, but the concepts of such a learning engine can be applied throughout the various rule engines of FIG. 4. Artificial intelligence, deep learning, machine learning, and recumbent learning may be applied as part of rule engines, and FIG. 10 is just one example of a learning engine.

In training engine 1002, historical reports 1012 are mined and converted into ML understandable report text 1014. The ML understandable text is run through machine learning algorithms (or other artificial intelligence, deep learning, or recumbent learning algorithms) to extract features 1016 related to radiology reports in this example. Then learning to identify a rule step 1018 takes rule 1010 and the extracted features 1016 and updates a classifier model 1026 based on the related learnings. In prediction engine 1004, the current report text 1020 is converted into ML understandable report text 1022. The ML understandable text is run through machine learning algorithms (or other artificial intelligence, deep learning, or recumbent learning algorithms) to extract features 1024. Classifier model 1026 takes the output of training engine 1002 and the current report features 1024 to apply and improve rules 1028. Rule 1028 is then fed back into 1010 to continue to be improved through the learning engine 1000. Thus, learning engine 1000 receives historical reports from said archive and uses machine learning to extract features from historical reports to provide update actions related to positive aspects of historical reports supplied from the feedback engine, in an embodiment.

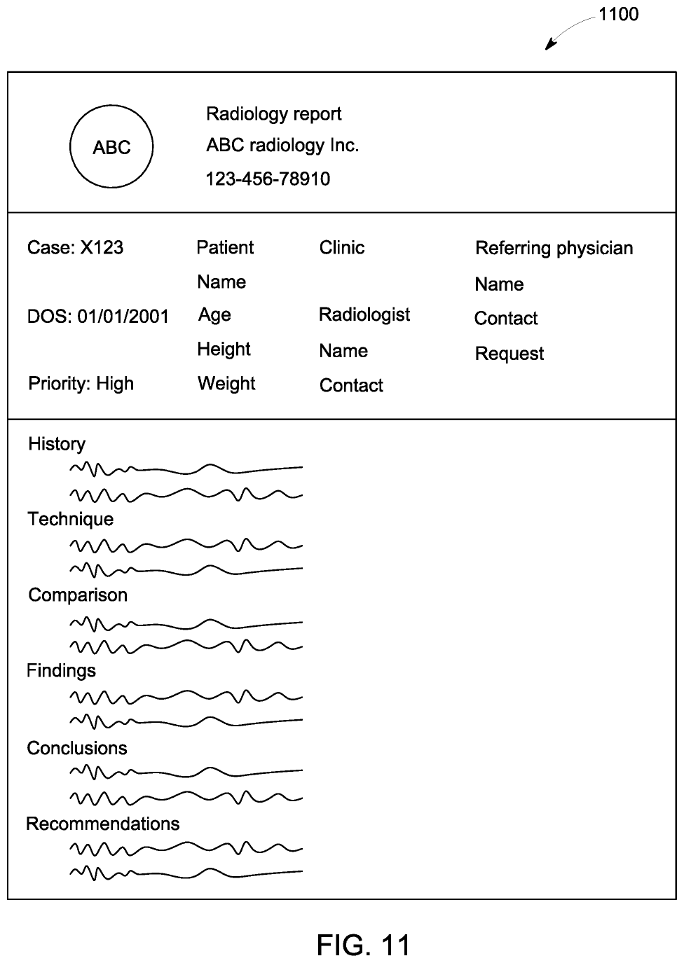

FIG. 11 shows an example report, according to an embodiment. Radiology report 1100 includes such sections as request details (case number, date of service [“DOS”], patient information, clinic information, referring physician information, etcetera), history, technique, comparison, findings (normal, abnormal or potentially abnormal), conclusions, and recommendations.

FIG. 12 shows a user interface for an improved reporting and collaboration technology, according to an embodiment. FIG. 12 includes reporting system 1200, in-progress report with user interface (“UI”) prompts 1202, improved report version 1204, UI prompt 1210, UI prompt 1212, and UI prompt 1214. In-progress report with UI prompts 1202 is an example of how the report may look while it is being generated. A user is putting text and information into the report and various engines are reviewing the input text along with report context to give on-the-fly reminders and prompts.

UI prompt 1210 sates “Omission of standard section, Section name: Technique.” UI prompt 1210 reminds the user that a standard section of a radiology report is missing, which may be generated by healthcare system guidelines engine 446, industry best practices engine 468, or another relevant engine depending on the embodiment. UI prompt 1212 states “Reporting style mismatch, No bullets in findings section.” UI prompt 1212 reminds the user that there are no bullets in the finding sections, which would make the report easier to read, which may be generated by human readable engine 470 or another relevant engine depending on the embodiment. UI prompt 1214 states “Omission of standard section, Section name: Impression.” UI prompt 1214 reminds the user that a standard section of a radiology report is missing, which may be generated by healthcare system guidelines engine 446, industry best practices engine 468, or another relevant engine depending on the embodiment. Improved report version 1204 is the final report where the user has improved the report based on these example prompts.

In the example of FIG. 12, the intent/situation analysis engine would note that the situation as a computed tomography (“CT”) scan of the abdomen and pelvis for right lower quadrant pain concerning for appendicitis. Process 400 is run during report generation and the rule engines checked for, among other things: (1) Omission of standard sections, where standard sections include: EXAM, HISTORY, TECHNIQUE, FINDINGS and IMPRESSION, (2) reporting style mismatch, expected reporting style is bullet points in Findings section, (3) reporting style mismatch, expected reporting style is bullet points in Impression section. Here the first and second rule evaluate to true in the respective rule engines and the configured action is applied in the report as an on-the-fly reminder. The evaluation for the third rule is configured to be “ignored” based on the outcome of the first rule. This is an example of a nested rule. If the first rule evaluated to be false, meaning “Impressions” was not omitted, then the third rule would be evaluated to check for bulleted style in impressions.

FIG. 13 shows another user interface for an improved reporting and collaboration technology, according to an embodiment. FIG. 13 includes reporting system 1300, in-progress report 1302, reminder 1304, error 1306, and expectation 1308. Reminder 1304 is a UI prompt that states “Reminder, to inform result to the patient.” Reminder 1304 may be generated by recipient preferences engine 460 in that the report should not only be provided to the referring physician but also the patient. In an alternative embodiment, reminder 1304 may be generated by cultural preferences engine 464 if the culture is one where the radiologist directly informs the patients. Error 1306 is a UI prompt that states “Missing insurance eligibility info, Context employer-sponsored lunch cancer screening missing info: Quit since.” Since this is an error of higher significance, the reporting system 1300 may highlight or underline the exact section related to the error to help the user and improve the user experience as well as save time in report editing. Error 1306 may be generated by financial requirements engine 444 to ensure that all information related to submitting a payment claim is included in the final report. Expectation 1308 is a UI prompt that states “Position mismatch, First section expected: Summary (or impression).” This means the system has an expectation of a certain layout that has not been realized yet in the current report. Expectation 1308 may be generated by healthcare system guidelines engine 446, industry best practices engine 468, or another relevant engine depending on the embodiment.

In the example of FIG. 13, process 400 is run and may check for the following, among other things: (1) Ordering of standard sections, where the order is, EXAM, HISTORY, TECHNIQUE, FINDINGS and IMPRESSION. This may be an institution or department wide rule on good documentation practice to ensure common standard across. This rule is “overridable” by conflicting rule by providing the reason. If the reason is provided, it can help help identify the right root cause of error violation during retrospection of historical work of radiologist. (2) Summary section in the beginning of the report if report length is greater than one page. The objective is to quickly classify screening. This is an example of position of a section of a report where the conventional ordering (Summary/Impression followed by Observation) as per the previously defined rule will have to be overridden based on the context. The context here is the intent of ordering the scan. Also, this is an example of a combination rule where additional condition check is on the length of the report. (3) Omission of insurance eligibility details in History section of the report (example is a 30-year history of smoking and current smoker or quit smoking in the last 15 years). This is an example of a rule configured for a non-medical stakeholder, to ensure details required to determine insurance eligibility is covered in the report. The rule can change over time and based on provider, hence needs detailed rule definition such as: (a) Is history section present? Yes. (b) Is insurance context “Employer-sponsored”? Yes. Then pull the relevant next rule set. (c) Does the report cover “number of pack-years”? Yes. (d) Does the report cover “current smoker”? No. (e) Does the report cover “quit smoking”? Yes. (f) Does the report have “when quit”? No. Then display configured error. There can be different flows based on previous condition. This is one illustration of rule evaluation and the rule evaluation logic can be optimized. There can also be provisioned for editing rules in the runtime with authorization workflow to promote the changes to production. (4) A culture-specific rule reminding that the radiologist is expected to convey the result, if screening is negative (normal result). This rule is activated based on the culture setting of the application, such as by cultural considerations engine 464. The rule checks the impression/summary to evaluate the screening result. If it is normal, then a standard information message is displayed. The UI prompts and on-the-fly reminders generated by the systems and methods herein can help in ensuring improved reports and report collaboration.

The benefits of systems and methods herein are both technical and practical to the reporting space. Reports are improved with less effort by report preparers. In the healthcare sphere, there is time saved, better care provided, money saved, and better communication among healthcare providers. Further, report preparers are further trained and supported in making improved reports.

The systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

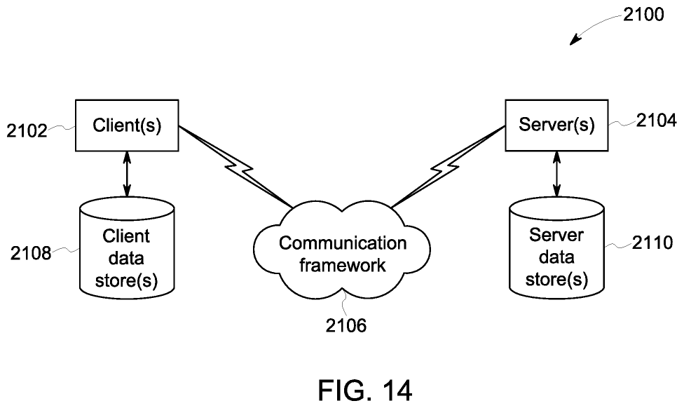

Referring to FIG. 14, there is illustrated a schematic block diagram of a computing environment 2100 in accordance with this disclosure in which the subject systems, methods and computer readable media can be deployed. The computing environment 2100 includes one or more client(s) 2102 (e.g., laptops, smart phones, PDAs, media players, computers, portable electronic devices, tablets, and the like). The client(s) 2102 can be hardware and/or software (e.g., threads, processes, computing devices). The computing environment 2100 also includes one or more server(s) 2104. The server(s) 2104 can also be hardware or hardware in combination with software (e.g., threads, processes, computing devices). The servers 2104 can house threads to perform transformations by employing aspects of this disclosure, for example. In various embodiments, one or more of the subject front end-components can be deployed as hardware and/or software at a client 2102 and one or more of the subject back-end components can be deployed as hardware and/or software at server 2104. One possible communication between a client 2102 and a server 2104 can be in the form of a data packet transmitted between two or more computer processes wherein the data packet may include video data. The data packet can include a metadata, e.g., associated contextual information, for example. The computing environment 2100 includes a communication framework 2106 (e.g., a global communication network such as the Internet, or mobile network(s)) that can be employed to facilitate communications between the client(s) 2102 and the server(s) 2104.

Communications can be facilitated via a wired (including optical fiber) and/or wireless technology. The client(s) 2102 include or are operatively connected to one or more client data store(s) 2108 that can be employed to store information local to the client(s) 2102 (e.g., associated contextual information). Similarly, the server(s) 2104 are operatively include or are operatively connected to one or more server data store(s) 2110 that can be employed to store information local to the servers 2104.

In one embodiment, a client 2102 can transfer an encoded file, in accordance with the disclosed subject matter, to server 2104. Server 2104 can store the file, decode the file, or transmit the file to another client 2102. It is to be appreciated, that a client 2102 can also transfer uncompressed file to a server 2104 and server 2104 can compress the file in accordance with the disclosed subject matter. Likewise, server 2104 can encode video information and transmit the information via communication framework 2106 to one or more clients 2102.

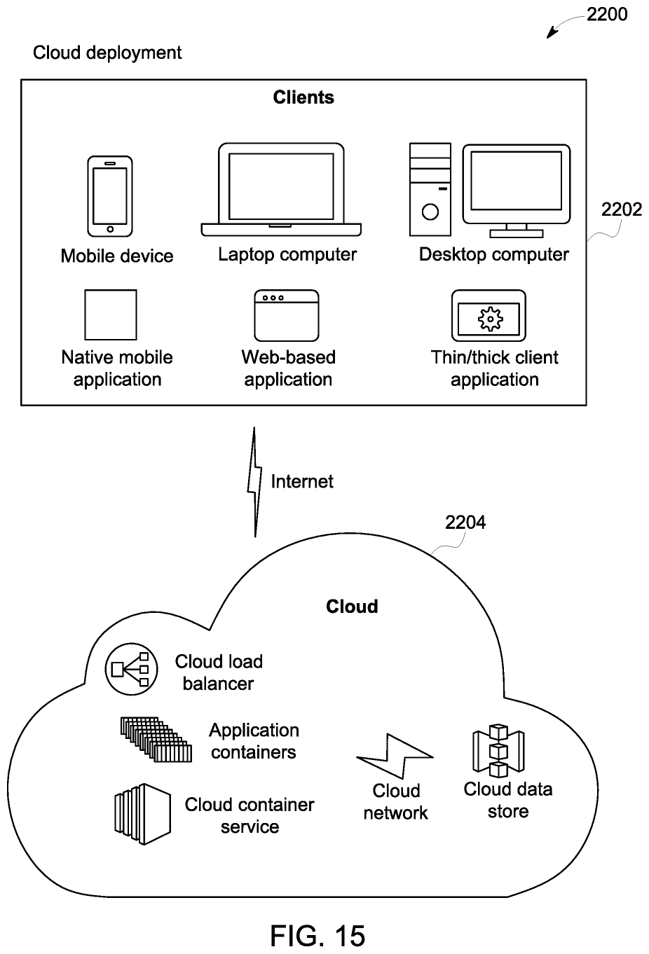

FIG. 15 illustrates a schematic block diagram of another example computing environment 2200 in accordance with this disclosure in which the subject systems, methods and computer readable media can be deployed. The computing environment 2200 includes a cloud deployment architecture consisting of one or more clients 2202 that can be communicatively coupled to a system cloud 2204 via a network (e.g., the Internet). The system cloud 2204 can include a cloud load balances, one or more application container, one or more cloud service containers, a cloud data store, and a cloud network that communicatively couples the one or more cloud components to the cloud data store. In accordance with the cloud deployment architecture, the clients 2202 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject native/reconstructed medical imaging systems deployed in the system cloud 2204. In various implementations, the one or more components of system 100 can be distributed between the clients 2202 and the system cloud 2204.

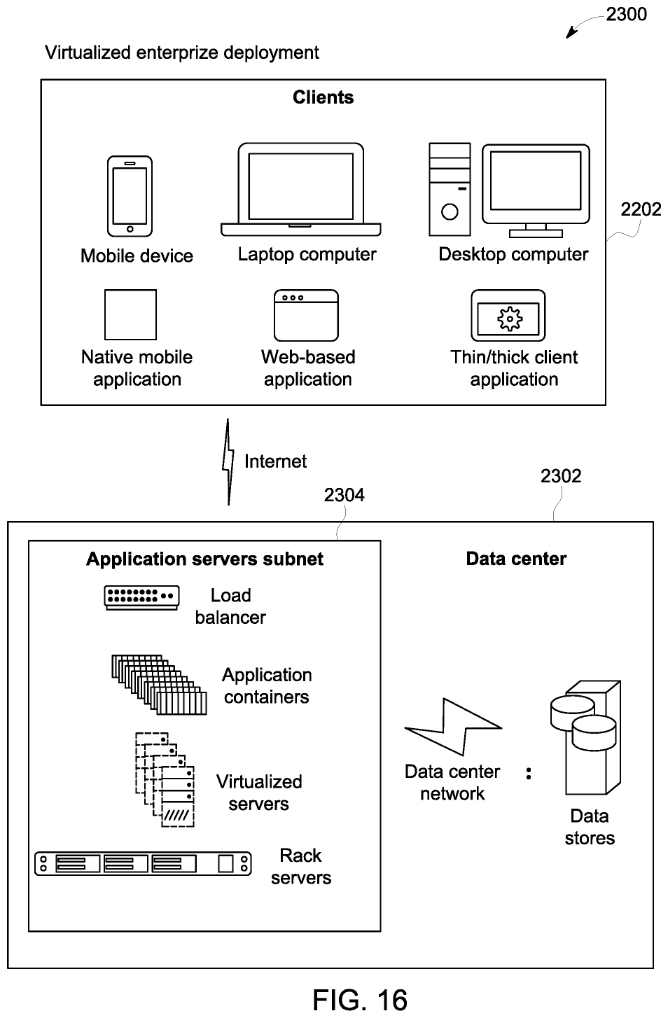

FIG. 16 illustrates a schematic block diagram of another example computing environment 2300 in accordance with this disclosure in which the subject systems (e.g., systems 100 and the like), methods and computer readable media can be deployed. The computing environment 2300 includes a virtualized enterprise deployment consisting of one or more clients 2202 that can be communicatively coupled to a remote data center 2302 via a network (e.g., the Internet). The remote data center 2302 can include an application servers subnet 2304 which can provide a load balancer, one or more application containers, one or more virtualized servers and one or more rack servers. The data center 2302 can also include one or more data stores that can be communicatively coupled to the application servers subnet 2304 via a data center network. In accordance with the virtualized enterprise deployment, the clients 2202 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject native/reconstructed medical imaging systems deployed in the data center 2302 and application servers subnet 2304. In various implementations, the one or more components of systems 100 can be distributed between the clients 2202 and the application servers subnet 2304 and the one or more data stores can be provided remotely at the data center 2302.

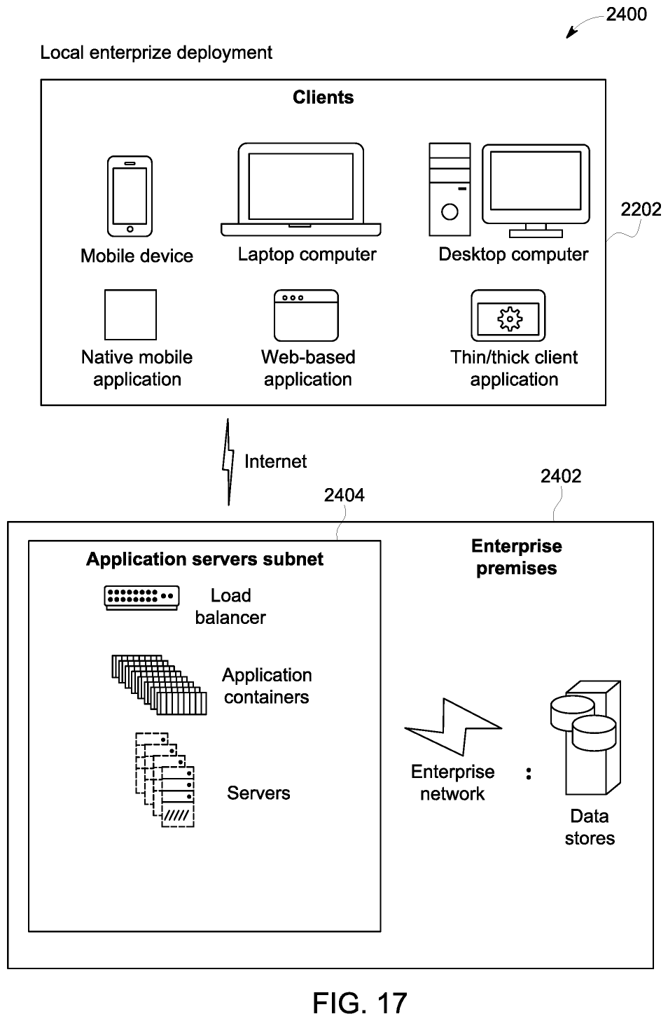

FIG. 17 illustrates a schematic block diagram of another example computing environment 2400 in accordance with this disclosure in which the subject systems, methods and computer readable media can be deployed. The computing environment 2400 includes a local enterprise deployment consisting of one or more clients 2202 that can be communicatively coupled to an application servers subnet 2404 via a network (e.g., the Internet). In accordance with this embodiment, the application servers subnet 2404 can be provided at the enterprise premises 2402 (e.g., as opposed to a remote data center 2302). The application servers subnet 2404 can include a load balancer, one or more application containers and one or more servers. The application servers subnet 2404 can be communicatively coupled to one or more data stores provided at the enterprise premises 2402 via an enterprise network. Similar to the cloud and virtualized enterprise deployments, the clients 2202 can include one or more clients devices (e.g., a mobile device, a laptop computer, a desktop computer, etc.) which can include or employ a suitable application (e.g., a native mobile application, a web-based application, a thin/thick client application, etc.) to access and employ one or more features and functionalities of the subject native/reconstructed medical imaging systems (e.g., system 100 and the like) deployed at the enterprise premises 2402 and the application servers subnet 2404. In various implementations, the one or more components of systems herein can be distributed between the clients 2202 and the application servers subnet 2404 and the one or more data stores can be provided at the enterprise premises 2402.

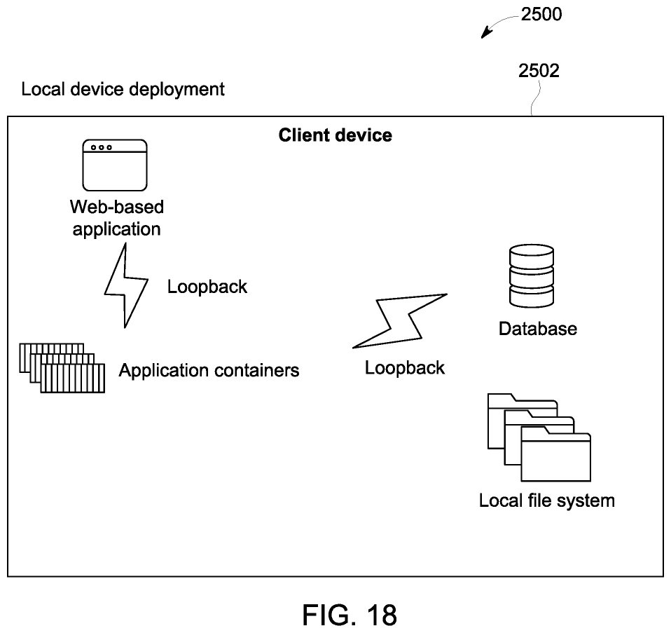

FIG. 18 illustrates a schematic block diagram of another example computing environment in accordance with this disclosure in which the subject systems, methods and computer readable media can be deployed. The computing environment includes a local device deployment in which all of the components of systems herein are provided at a single client device 2502. With this implementation, the client device 2502 can include a web-based application which can be communicatively coupled via a loopback to one or more application containers. The one or more application containers can be communicatively coupled via a loopback to one or more databases and/or one or more local file systems.

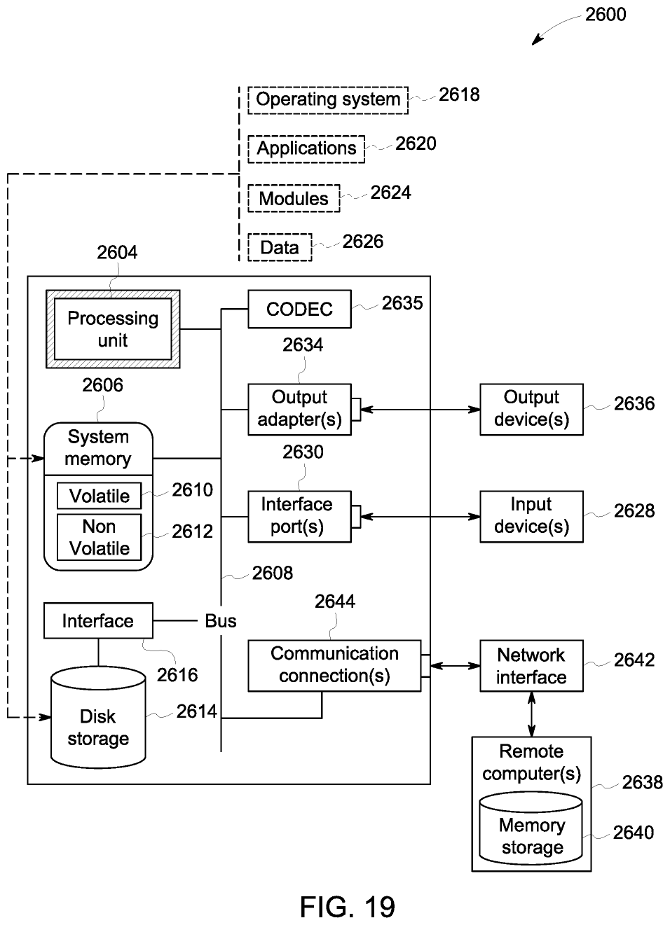

With reference to FIG. 19, a suitable environment 2600 for implementing various aspects of the claimed subject matter includes a computer 2602. The computer 2602 includes a processing unit 2604, a system memory 2606, a codec 2605, and a system bus 2608. The system bus 2608 couples system components including, but not limited to, the system memory 2606 to the processing unit 2604. The processing unit 2604 can be any of various available processors. Dual microprocessors and other multiprocessor architectures also can be employed as the processing unit 2604.

The system bus 2608 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 22104), and Small Computer Systems Interface (SCSI).

The system memory 2606 includes volatile memory 2610 and non-volatile memory 2612. The basic input/output system (BIOS), containing the basic routines to transfer information between elements within the computer 2602, such as during start-up, is stored in non-volatile memory 2612. In addition, according to present innovations, codec 2605 may include at least one of an encoder or decoder, wherein the at least one of an encoder or decoder may consist of hardware, a combination of hardware and software, or software. Although, codec 2605 is depicted as a separate component, codec 2605 may be contained within non-volatile memory 2612. By way of illustration, and not limitation, non-volatile memory 2612 can include read only memory (ROM), programmable ROM (PROM), electrically programmable ROM (EPROM), electrically erasable programmable ROM (EEPROM), or flash memory. Volatile memory 2210 includes random access memory (RAM), which acts as external cache memory. According to present aspects, the volatile memory may store the write operation retry logic and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM).

Computer 2602 may also include removable/non-removable, volatile/non-volatile computer storage medium. FIG. 19 illustrates, for example, disk storage 2614. Disk storage 2614 includes, but is not limited to, devices like a magnetic disk drive, solid state disk (SSD) floppy disk drive, tape drive, Zip drive, flash memory card, or memory stick. In addition, disk storage 2614 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 2614 to the system bus 2608, a removable or non-removable interface is typically used, such as interface 2616.

It is to be appreciated that FIG. 16 describes software that acts as an intermediary between users and the basic computer resources described in the suitable operating environment 2600. Such software includes an operating system 2618. Operating system 2618, which can be stored on disk storage 2614, acts to control and allocate resources of the computer system 2602. Applications 2620 take advantage of the management of resources by operating system 2618 through program modules 2624, and program data 2626, such as the boot/shutdown transaction table and the like, stored either in system memory 2606 or on disk storage 2614. It is to be appreciated that the claimed subject matter can be implemented with various operating systems or combinations of operating systems.

A user enters commands or information into the computer 2602 through input device(s) 2628. Input devices 2628 include, but are not limited to, a pointing device such as a mouse, trackball, stylus, touch pad, keyboard, microphone, joystick, game pad, satellite dish, scanner, TV tuner card, digital camera, digital video camera, web camera, microphone, and the like. These and other input devices connect to the processing unit 2604 through the system bus 2608 via interface port(s) 2630. Interface port(s) 2630 include, for example, a serial port, a parallel port, a game port, and a universal serial bus (USB). Output device(s) 2636 use some of the same type of ports as input device(s). Thus, for example, a USB port may be used to provide input to computer 2602, and to output information from computer 2602 to an output device 2636. Output adapter 2634 is provided to illustrate that there are some output devices 2636 like monitors, speakers, and printers, among other output devices 2636, which require special adapters. The output adapters 2634 include, by way of illustration and not limitation, video and sound cards that provide a means of connection between the output device 2636 and the system bus 2608. It should be noted that other devices and/or systems of devices provide both input and output capabilities such as remote computer(s) 2638.

Computer 2602 can operate in a networked environment using logical connections to one or more remote computers, such as remote computer(s) 2638. The remote computer(s) 2638 can be a personal computer, a server, a router, a network PC, a workstation, a microprocessor based appliance, a peer device, a smart phone, a tablet, or other network node, and typically includes many of the elements described relative to computer 2602. For purposes of brevity, only a memory storage device 2640 is illustrated with remote computer(s) 2638. Remote computer(s) 2638 is logically connected to computer 2602 through a network interface 2642 and then connected via communication connection(s) 2644. Network interface 2642 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 2644 refers to the hardware/software employed to connect the network interface 2642 to the bus 2608. While communication connection 2644 is shown for illustrative clarity inside computer 2602, it can also be external to computer 2602. The hardware/software necessary for connection to the network interface 2642 includes, for exemplary purposes only, internal and external technologies such as, modems including regular telephone grade modems, cable modems and DSL modems, ISDN adapters, and wired and wireless Ethernet cards, hubs, and routers.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from any computer-readable device or storage media

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

Moreover, because at least employing a convolutional neural network, etc. is established from a combination of electrical and mechanical components and circuitry, a human is unable to replicate or perform processing performed rule learning engine 218, rule engine 306, and the engines of FIG. 4, disclosed herein. For example, a human is unable to perform machine learning associated with a convolutional neural network, etc.

In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A system for generating a report, comprising:
an image viewer and reporting system for displaying report information and receiving user input; and
a reporting assistant that receives and analyzes said report information and user input in comparison to at least one rule engine to generate a report update action, and providing the report update action to the image viewer and reporting system while the user is currently using the image viewer and reporting system.

2. The system of claim 1, wherein:
the at least one rule engine is a learning engine.

3. The system of claim 2, further comprising:
a feedback engine;
an archive with historical reports; and
wherein said learning engine receives historical reports from said archive and uses machine learning to extract features from historical reports to provide update actions related to positive aspects of historical reports supplied from the feedback engine.

4. The system of claim 1, wherein:
the report information includes radiology information; and
the image viewer and reporting system displays a radiology image.

5. The system of claim 4, wherein the at least one rule engines comprises:
a recipient preference engine that analyzes the report information in comparison with the preferences of the intended recipient and generates a report update action including a recommendation for altering the report to fit with the preferences of the intended recipient.

6. The system of claim 5, wherein:
the recipient is a patient or a referring physician.

7. The system of claim 1, wherein:
said analyzing includes a requirements evaluation in comparison to at least one of a legal, financial, healthcare system, industry, care based, or safety requirements.

8. The system of claim 1, wherein said analyzing to generate a report update action includes the steps of:
analyzing user input and report information to identify report recommendations;
performing a requirements evaluation comparing user input and report information with report requirements to identify rule violations;
evaluating user input and report information to identify useful output recommendations based on recipient information; and
providing an update action to the image viewer and reporting system, said update action including at least one of a report recommendation, rule violation, or useful output recommendation.

9. The system of claim 1, wherein:
the report information is radiology report information; and
the at least one rule engine is a wording and vocabulary engine that analyzes the radiology report information for at least one of double negatives, defensive posturing, ambiguous vocabulary, hedge vocabulary, modifiers such as quantitative adjectives, and generalizations.

10. The system of claim 1, wherein the at least one report engine comprises:
at least one rule engine related to receiving input;
at least one rule engine related to processing and analyzing input;
at least one rule engine related to requirements evaluation; and
at least one rule engine related to useful output evaluation.

11. A method for generating a report, comprising the steps of:
receiving user input and report information from an image viewer and reporting system;
analyzing user input and report information to identify report recommendations;
performing a requirements evaluation comparing user input and report information with report requirements to identify rule violations;
evaluating user input and report information to identify useful output recommendations based on recipient information; and
providing an update action to the image viewer and reporting system, said update action including at least one of a report recommendation, rule violation, or useful output recommendation.

12. The method of claim 11, further comprising the step of:
displaying said update action on a user interface of the image viewer and reporting system.

13. The method of claim 12, further comprising the steps of:
receiving user input response in response to the displaying of the update action on the user interface of the image viewer and reporting section; and
modifying the report information based on said user input response.

14. The method of claim 11, wherein:
said update action is a useful output recommendation and said update action is applied automatically to the report without user interaction.

15. The method of claim 11, wherein:
each of the receiving, analyzing, performing, and evaluating steps include at least one rule engine to process the report information and output update actions.

16. A non-transitory computer readable storage medium having stored thereon a computer program comprising instructions, which, when executed by a computer, cause the computer to execute the actions of:
receiving user input and report information from an image viewer and reporting system;
analyzing user input and report information to identify report recommendations;
performing a requirements evaluation comparing user input and report information with report requirements to identify rule violations;
evaluating user input and report information to identify useful output recommendations based on recipient information; and
providing an update action to the image viewer and reporting system, said update action including at least one of a report recommendation, rule violation, or useful output recommendation.

17. The computer readable storage medium of claim 16, wherein the instructions further cause the computer to:
display said update action on a user interface of the image viewer and reporting system.

18. The computer readable storage medium of claim 17, wherein the instructions further cause the computer to:
receive user input response in response to the displaying of the update action on the user interface of the image viewer and reporting section; and
modify the report information based on said user input response.

19. The computer readable storage medium of claim 16, wherein:
said update action is a useful output recommendation and said update action is applied automatically to the report without user interaction.

20. The computer readable storage medium of claim 16, wherein:
each of the receiving, analyzing, performing, and evaluating steps include at least one rule engine to process the report information and output update actions.
